(19) United States
(12) Patent Application Publication
(10) Pub. No.: US 2022/0233738 A1
(43) Pub. Date: Jul. 28, 2022

(54) Air Sterilizer

(71) Applicant: RIP JAPAN CO., LTD., Tokyo (JP)

(72) Inventor: Kensaku YUKINO, Tokyo (JP)

(21) Appl. No.: 17/579,562

(22) Filed: Jan. 19, 2022

(30) Foreign Application Priority Data
Jan. 28, 2021 (JP) 2021-011652

(51) Int. Cl. A61L 9/20

(52) U.S. Cl. CPC A61L 9/20; A61L 2209/11; A61L 2209/12

(57) ABSTRACT

An air sterilizer to effectively prevent infection of diseases is provided. The air sterilizer includes a case having a suction port and a discharge port of air, a sterilizer arranged inside the case and sterilizing or inactivating bacteria or viruses in air inside the case, and an airflow generator generating an airflow that directs air containing at least an exhalation of a person in a space outside of the case to the suction port.

### TECHNICAL FIELD

The present invention relates to an air sterilizer.

### BACKGROUND ART

In recent years, the spread of COVID-19 has had a major impact on the world and has had a serious impact on the national economies. The droplets generated in a mouth and nasal cavity of a person during coughing, sneezing, talking, and singing are released from the mouth, and large droplets are scattered about 2 meters, but they fall afterwards. Droplets smaller than 5 micrometers in diameter and their dried droplet nuclei are called aerosols, can remain airborne for long periods of time, and cause the spread of infectious diseases such as COVID-19 (novel coronavirus).

As a countermeasure against this, an air sterilizer using a germicidal lamp or an ultraviolet LED (light emitting diode) as shown in FIG. 1, for example, has been developed and sold. However, since these air sterilizers are located independently of sources of aerosols containing bacteria or viruses, i.e., the location of the infected person, an exhaled air of a person containing aerosols does not always pass through the air sterilizer. In the drawing, an air sterilizer 1 is arranged on a back side of people who are facing each other and having conversations. The air sucked from a suction port 2 is sterilized inside and then horizontally discharged from a discharge port 3. A pathogen 5 discharged from an exhaled air of an infected person 4 is carried by an air flow 6 discharged from the discharge port 3 to a person 7 facing each other, and this person may be infected. In such an arrangement, the pathogen 5 enters the suction port 2 of the air sterilizer 1 after going through the room, and it is only a small portion of the pathogen discharged from the infected person 4. Therefore, there is a risk that the aerosol released from the infected person is spread out the room by the air flow generated by the air sterilizer without being inactivated.

### CITATION LIST

### Patent Document

For example, Patent Document 1 discloses an apparatus which is installed on both sides of a bed in a hospital room, sterilizes intake air with ultraviolet rays and blows it upward like an air curtain.

Patent Document 1: U.S. Pat. No. 9,310,088 B2

### SUMMARY OF INVENTION

### Technical Problem

The apparatus disclosed in Patent Document 1 is intended to intake air beside the bed, and it is not possible to suck the exhalation of the infected person intensively. Therefore, an object of the present invention is to effectively prevent infection of a disease by sucking the exhalation of the infected person.

### SOLUTION TO PROBLEM

In order to achieve the above objects, an air sterilizer according to one aspect of the present invention includes a case having a suction port and a discharge port of air, a sterilizer arranged inside the case and sterilizing or inactivating bacteria or viruses in air inside the case, and an airflow generator generating an airflow that directs air containing an exhalation of a person in a space outside of the case to the suction port.

The suction port, in an installation condition, may be arranged at an upper region of a space where the person resides and opens at a lower region.

The suction port may be provided with a cylindrical rectifying member in which an inner diameter increases toward an end part.

The airflow generator may be arranged inside the case.

The air sterilizer may further include a duct connecting to the discharge port and extending below the suction port, and the duct may have a vent hole facing the suction port.

The airflow generator may be arranged outside of the case and at a position facing the suction port.

The air sterilizer may further include a transparent panel connecting to a lower part of the suction port and extending downward.

The air sterilizer may include a top plate on which the suction port is formed, a body of a lectern supporting the top plate, and a shield connecting to an upper part of the top plate and extending upward toward the top plate, and the discharging port may open below the top plate.

### Effect of the Invention

According to the present invention, infection of diseases can be effectively prevented.

### BRIEF DESCRIPTION OF DRAWINGS

FIG. 1 is a schematic diagram of a conventional air sterilizer.

FIG. 2A is a schematic front view of an air sterilizer according to a first embodiment of the present invention.

FIG. 2B is a schematic cross-sectional view taken along line A-A in FIG. 2A.

FIG. 3A is a schematic front view of an air sterilizer according to a second embodiment of the present invention.

FIG. 3B is a view as seen from a direction of an arrow B in FIG. 3A.

FIG. 4 is a schematic front view of an air sterilizer according to a third embodiment of the present invention.

FIG. 5A is a schematic front view of an air sterilizer according to a fourth embodiment of the present invention.

FIG. 5B is a schematic right-side view of the air sterilizer according to the fourth embodiment of the present invention.

FIG. 6 is a schematic side view of an air sterilizer according to a fifth embodiment of the present invention.

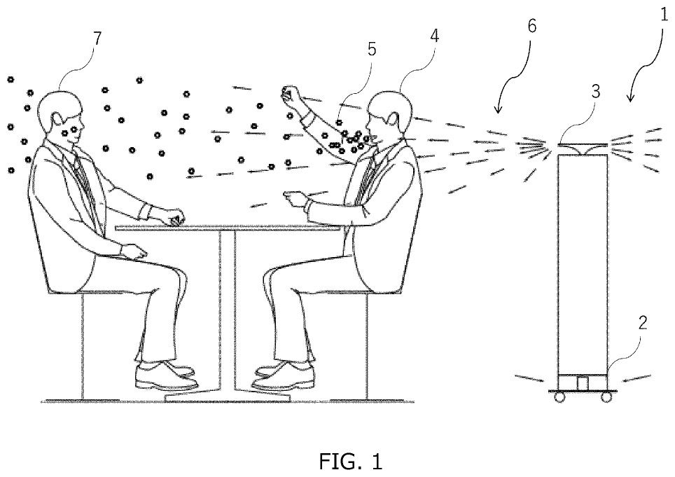

### PREFERRED EMBODIMENT

An air sterilizer according to an embodiment of the present invention will be described below with reference to the drawings. The air sterilizer is a device to suck exhalation of a facing speaker, sterilize or inactivate bacteria or viruses in an air, and discharge.

### Air Sterilizer (1)

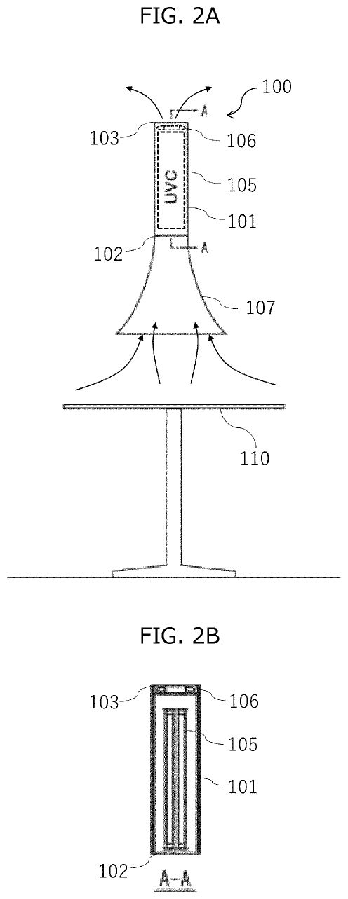

As illustrated in FIG. 2A and FIG. 2B, the air sterilizer 100 according to a first embodiment of the present invention has a box-shaped case 101. The case 101 may have a substantially cylindrical shape in addition to a substantially rectangular parallelepiped shape. An inner peripheral surface of the case 101 has a reflective surface which efficiently reflects ultraviolet rays emitted by a sterilizer 105 arranged inside. Alternatively, an outer peripheral surface of the case 101 may be processed so as not to transmit ultraviolet rays, or the case 101 may be formed of a member that does not transmit ultraviolet rays.

The case 101 has a suction port 102 and a discharge port 103 of air. The suction port 102 is arranged at the upper region of the space where a person resides and opens at a lower region. More specifically, the suction port 102 is, for example, arranged between speakers facing each other and at the upper region of the space, and the suction port 102 is opened toward a space between the speakers. The case 101 is, for example, suspended above a table 110, and the suction port 102 is arranged at lower part of the case 101. The air sterilizer 100 may have a self-supporting support post on the table 110 or on a floor surface, and the support post may support the case 101 so that the suction port 102 is held above the table 110.

The exhalations of the speakers have higher temperatures than room temperatures so it has a property of moving upward. Therefore, according to a configuration in which the suction port 102 is opened on the upper region of the speakers, the exhalations of the speakers can be efficiently collected.

The discharge port 103 for the sucked air is opened in a direction different from the suction port 102. The discharge port 103 is formed at a position across the sterilizer 105 and opposite to the suction port 102. In the present embodiment, the discharge port 103 is opened toward the upper region in the upper part of the case 101, but it is not limited to this embodiment.

The air sterilizer 100 has the sterilizer 105, an airflow generator 106, and a rectifying member 107 as a configuration for sterilizing or inactivating air.

The sterilizer 105 is a device that is arranged inside the case 101 and emits ultraviolet rays to an inside of the case 101 to sterilizer or inactivate bacteria or viruses contained in the air sucked into the case 101. As illustrated in FIG. 2B, in the present embodiment, the sterilizer 105 is constituted by a pair of germicidal lamps which are elongated in a flow path direction. Further, the sterilizer 105 may also use a sterilizing power of photocatalysts excited by ultraviolet rays in addition to the sterilizing power of the ultraviolet rays themselves by arranging the photocatalysts in surroundings.

The airflow generator 106 is a device that is generating an air flow from an outside of the case 101 to an inside of the case 101. The airflow generator 106 is, for example, a fan rotated by a motor. Further, quietness can be ensured by using a cross flow fan for the airflow generator 106.

In the present embodiment, the airflow generator 106 is arranged inside the case 101 and between the sterilizer 105 and the discharge port 103. The airflow generator 106 may be located on a flow path virtually connecting the suction port 102, the sterilizer 105, and the discharge port 103, and may be arranged between the suction port 102 and the sterilizer 105, for example. The air sucked from the suction port 102 is sterilized or inactivated by the sterilizer 105 and discharged to the outside of the case 101 by the air flow generated by the air flow generating device 106.

The rectifying member 107 is a member arranged at the suction port 102. The rectifying member 107 is a cylindrical member having an inner diameter that increases from the suction port 102 toward an end, that is, a trumpet-shaped member. The rectifying member 107 guides the air in the lower region of the air sterilizer 100 to the suction port 102. The rectifying member 107 may be integrated with the case 101.

In general, a fan as the airflow generator 106 generates an air flow having momentum and directivity on a discharging side, but has a characteristic of sucking surrounding air from all directions on a sucking side. Therefore, if an inhalation is performed by a fan, not only the exhaled air of the person seated at a lower region but also the air from all direction in a vicinity of the suction port 102 is sucked. Thus, it is difficult to suck all of the exhaled air. On the other hand, according to the air sterilizer 100 having the rectifying member 107, the rectifying member 107 can mainly suck an air of a lower region of the suction port 102. As a result, the exhaled air of the person seated at the lower region of the air sterilizer 100 can be sucked more efficiently. In other words, since released pathogens included in human breath, sneeze, cough and the like can be inactivated without spreading in a room, infection of disease can be prevented more effectively than the conventional sterilizer.

### Air Sterilizer (2)

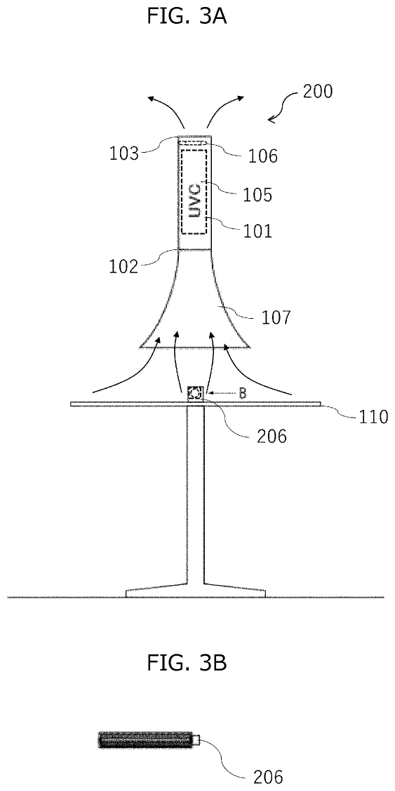

With reference to FIG. 3A and FIG. 3B, an air sterilizer 200 according to a second embodiment of the present invention will be described with focus on parts different from those of the first embodiment. This air sterilizer 200 has an airflow generator 206 that is arranged outside the case 101 and at a position facing the suction port 102 and blows out an air flow having directivity upward. More specifically, the airflow generator 206 is arranged on the top plate of the table 110 placed below the air sterilizer 200. In the following description, the same components as those of the other embodiments are denoted by the same reference numerals.

As illustrated in FIG. 3B, the airflow generator 206 is, for example, a cross-flow fan. An outer shape of the airflow generator 206 is, for example, substantially rectangular parallelepiped shape and arranged so as to separate speakers facing each other. For example, the airflow generator 206 is arranged in a substantially center of the table 110 and substantially parallel to edges of the table 110. The airflow generator 206 may have a cover. The cover can ensure safety, for example, by preventing a person's hand from directly touching the fan. The cover may also be waterproof or water repellent. According to this configuration, it is possible to prevent the fan from getting dirty even when liquid or the like is spilled on the table 110.

The airflow generator 206 can be arranged in place of or in addition to the airflow generator 106 arranged inside the case 101. By arranging each of the airflow generators 106 and 206 before and after the sterilizer 105 on the flow path direction of the air flow, the airflow generators 106 and 206 form a push-pull type device which forms a uniform captured air flow. According to this configuration, an upward airflow becomes more stable and stronger, and the exhalation of the speakers facing each other across the table 110 can be more efficiently guided to the suction port 102.

### Air Sterilizer (3)

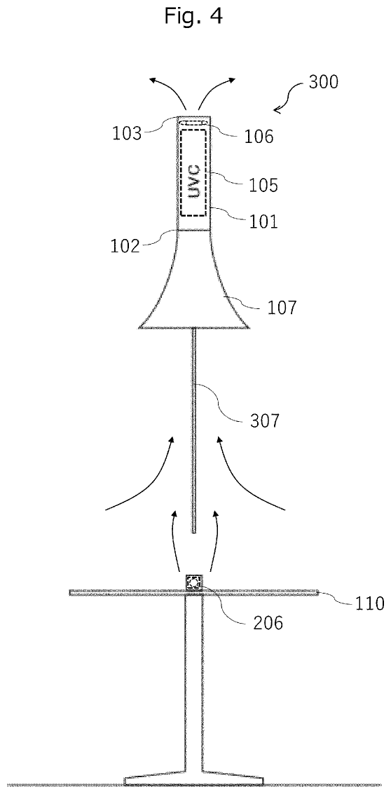

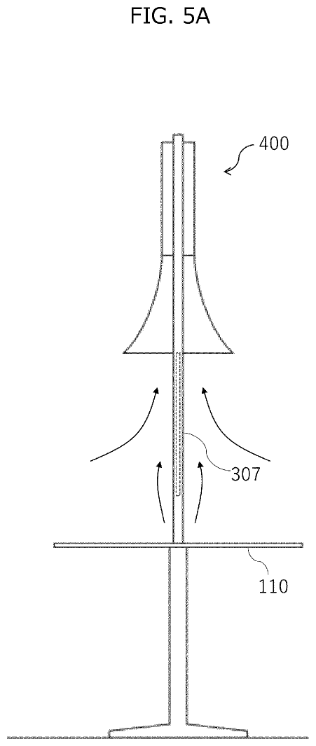

As illustrated in FIG. 4, an air sterilizer 300 according to a third embodiment of the present invention includes a transparent panel 307 which is connected to a lower part of the suction port 102 and extends downward. The transparent panel 307 is, for example, an acrylic plate, and arranged so as to block an exhalation of a person facing the table 110. According to this configuration, it is possible to prevent large droplets contained in the exhalation of the speaker from reaching a person facing the speaker even if the droplets do not get on the air flow from a lower region to an upper region. Further, according to the transparent panel 307, even when an upward air flow is disturbed by another blower such as an air conditioner or a ventilation system, the air flow can be stabilized.

A predetermined gap is provided between the transparent panel 307 and the airflow generator 206 arranged on the table 110. According to this configuration, one airflow generator 206 can generate an upward air flow on both side of the transparent panel 307.

In the above-described embodiments, two airflow generators 106 and 206, and one sterilizer 105 are provided, but the air sterilizer according to the present invention may include more airflow generators and sterilizers than those described. For example, two sets in total may be arranged on one side and another side of the transparent panel.

### Air Sterilizer (4)

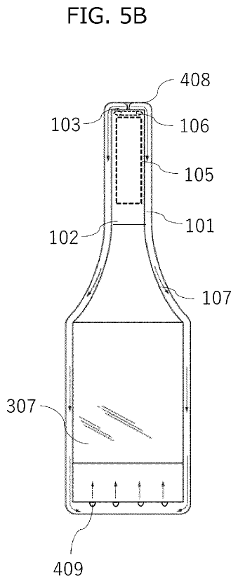

With reference to FIG. 5A and FIG. 5B, an air sterilizer 400 according to a fourth embodiment of the present invention will be described with focus on parts different from those of the third embodiment. This air sterilizer 400 includes a duct 408 connected to the discharge port 103 and extends below the suction port 102. The duct 408 is a hollow member and air discharged from the discharge port 103 flows inside. Further, the duct 408 has an annular shape and both ends are connected to the discharge port 103. The duct 408 is fixed to the case 101 and the rectifying member 107 and along sides of the transparent panel 307. Furthermore, the duct 408 is connected to the top plate of the table 110 below the suction port 102.

In the duct 408, a vent hole 409 is bored in a direction facing the suction port 102 in a top surface part of the table 110. The air flowing into the duct 408 from the discharge port 103 blows out from the vent hole 409 to generate an upward air flow above the table 110. The vent hole 409 may be a nozzle.

According to this configuration, since the exhalation can be efficiently collected by the air flow from the vent hole 409 and it can be formed more compactly than a configuration in which a separate airflow generator is placed on the table 110, the table 110 can be effectively used when eating or the like.

### Air Sterilizer (5)

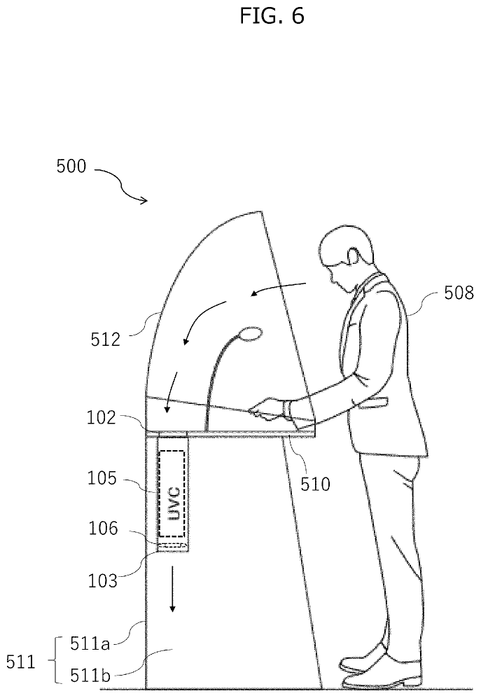

With reference to FIG. 6, an air sterilizer 500 according to a fifth embodiment of the present invention will be described with focus on parts different from those of the first embodiment. In the present embodiment, the air sterilizer is different from the embodiment described above in that the air sterilizer is configured as a lectern in which the suction port 102 is formed on the top plate 510.

The top plate 510 is a member to form a top surface of the lectern and a microphone or the like that collects the voice of a speaker 508 may be placed on the top plate 510.
A through hole having a shape corresponding to the suction port 102 is formed on the top plate 510, and the suction port 102 is connected to this through hole.
   The top plate 510 is supported by a body of the lectern in which a front panel 511a and left and right side-panels 511b are arranged in a U shape. The front panel 511a is, for example, made of an opaque panel so as to hide a lower body of a performer. Further, the left and right side-panels 511b may also be made of opaque panels. In the drawing, for convenience, configurations of the suction port 102, the discharge port 103, the sterilizer 105 and the airflow generator 106 arranged on a back side of the left and right side-panels 511b are illustrated. The discharge port 103 is formed on a lower surface side of the top plate 510 and opens below the top plate 510, and the air in which pathogen is inactivated is discharged below the top plate 510.

A shield 512 extending upward from the top plate 510 is connected to an upper part of the top plate 510. The shield 512 is, for example, a member connected near one end of the top plate 510 and curved so as to cover the top plate 510. Further, the shield 512 is a transparent member. According to this configuration, exhalation of the speaker 508 standing on another end side of the lectern and speaks toward the shield 512 can be blocked by the shield 512, thereby infection to the audience can be prevented. Further, since the shield 512 can guide the air flow generated by the airflow generator 106 in a direction from the speaker to the suction port 102, the exhalation of the speaker 508 can be sucked more efficiently. In other words, according to this lectern type of the air sterilizer 500, the speaker or the performer can prevent spreading pathogen into the room without using surgical masks. Therefore, contents of the speech are clarified and facial expressions of the speaker or the performer are not covered by the surgical masks, so effective press conferences or lectures can be held.

According to the air sterilizer of the present embodiment described above, it is possible to inactivate the pathogen released from the exhalation of the person without spreading in the room, so that the infection of the disease can be effectively prevented. Although the embodiments described above have described modes of inhaling pathogens released from a person sitting on a table and a person standing toward the lectern, the object of the present invention is not limited to these and can be widely applied to a person standing in front of a counter at a hospital reception or a person coming for an examination.

### REFERENCE SIGNS LIST

100 air sterilizer
101 case
102 suction port
103 discharge port
105 sterilizer
106 airflow generator
107 rectifying member
110 table

What is claimed is:

1. An air sterilizer comprising:
a case having a suction port and a discharge port of air;
a sterilizer arranged inside the case and sterilizing or inactivating bacteria or viruses in air inside the case; and
an airflow generator generating an airflow that directs air containing an exhalation of a person in a space outside of the case to the suction port.

2. The air sterilizer according to claim 1, wherein the suction port, in an installation condition, is arranged at an upper region of a space where the person resides and opens at a lower region.

3. The air sterilizer according to claim 1, wherein the suction port is provided with a cylindrical rectifying member in which an inner diameter increases toward an end part.

4. The air sterilizer according to claim 1, wherein the airflow generator is arranged inside the case.

5. The air sterilizer according to claim 4, further comprising a duct connecting to the discharge port and extending below the suction port, wherein the duct has a vent hole facing the suction port.

6. The air sterilizer according to claim 1, wherein the airflow generator is arranged outside of the case and at a position facing the suction port.

7. The air sterilizer according to claim 1, further comprising a transparent panel connecting to a lower part of the suction port and extending downward.

8. The air sterilizer according to claim 1, comprising:
a top plate on which the suction port is formed;
a body of a lectern supporting the top plate;
a shield connecting to an upper part of the top plate and extending upward toward the top plate;
wherein the discharging port opens below the top plate.
